(19) United States
(12) Patent Application Publication
(10) Pub. No.: US 2011/0301405 A1
(43) Pub. Date: Dec. 8, 2011

(54) SLEEPING INDUCER

(76) Inventor: Yunhyeong Cho, Seoul (KR)

(21) Appl. No.: 13/041,652

(22) Filed: Mar. 7, 2011

(30) Foreign Application Priority Data
Jun. 4, 2010 (KR) 10-2010-0052652

(51) Int. Cl. A61M 21/00

(52) U.S. Cl. 600/27

(57) ABSTRACT

Disclosed is a sleeping inducer, wherein the sleeping inducer is configured to move along like a lung that is shrunk and expanded in response to the respiration when a person respires during sleep, and includes a unit capable of vertically moving inside a doll that is held during sleep or a pillow on which a head is laid during sleep, to thereby induce a sleep state just like being hugged and slept by a mom.

### CROSS REFERENCE TO RELATED APPLICATIONS

Pursuant to 35 U.S.C. §119 (a), this application claims the benefit of earlier filing date and right of priority to Korean Patent Application No. 10-2010-0052652, filed on Jun. 4, 2010, the contents of which is hereby incorporated by reference in their entirety.

### BACKGROUND OF THE DISCLOSURE

1. Technical Field

The present disclosure relates to a sleeping inducer, and more particularly to a sleeping inducer capable moving like a lung that is shrunk and expanded in response to respiration when a person respires during sleep, and still more particularly to a sleeping inducer that includes a means capable of vertically moving inside a doll that is held during sleep or a pillow on which a head is laid during sleep, to thereby induce a sleep state just like being hugged and slept by a mom.

2. Background Art

Generally, people sleep on a pillow, and an infant and a child is provided with a comforting and soothing environment or device such as a doll by which the infant and the child can be easily lulled to sleep.

Devices are known in the art to help lull infants to sleep. Typically, these devices promote an atmosphere of well being for infants, helping them fall asleep faster and remain asleep for longer periods of time.

In view of the above fact, there has been proposed many devices and functions such as pillows and dolls to help lull infants or induce people to sleep.

One example of the conventional art has been disclosed in Korea Patent Laid-Open Publication No. 2006-0097797 in which a pillow including a sound device generating heart pulsation sound using a speaker is proposed. Another example of the conventional art has been disclosed in Korea Utility Model Patent No. 20-0194426 in which a pillow including a motor generating a vibration similar to heart pulsation is described.

However, the heart pulsation sound generated by the sound device is disadvantageous in that the sound is just a mechanical sound to become an impediment to sleep. The vibration generated by the motor is also disadvantageous in that the vibration is a simple monotonous stimulus that is repeated in a short period of time, and cannot provide a comfort and reality effect like those of a mother's bosom, although the vibration is generated 70 times per minute and known to provide a stimulus similar to that of a heart pulsation period.

That is, the conventional art is disadvantageous in that vibration transmitted by a motor is a simple trembling, such that users not accustomed thereto can be provided with a disapproval and, as a result an effective sleep cannot be sufficiently induced.

### SUMMARY OF THE DISCLOSURE

An object of the present disclosure is to solve at least one or more of the above problems and/or disadvantages in a whole or in part and to provide at least the advantages in which sleep state like that in a mother's bosom is induced using a pillow to sleep on or a doll that is hugged during sleep.

That is, the present disclosure is to provide a sleep inducer configured to provide a sleep state excellent in comfort and reality effect like those of a mother’ bosom and to effectively and sufficiently induce a sleep to a user, an infant or a child easily distracted by noises or erratic sounds, which make promoting adequate sleep difficult.

In order to achieve at least the above objects, in whole or in part, and in accordance with the purposes of the invention, as embodied and broadly described, and in one general aspect of the present invention, there is provided a sleeping inducer, the sleeping inducer comprising: a cover; a moving plate included inside the cover and disposed underneath the cover; and moving means repeatedly and vertically moving the moving plate.

Preferably, the moving plate is distanced from the cover in the range of a gap of 0.1 cm to 5.0 cm.

Preferably, the moving plate is closely abutted to the cover.

Preferably, the moving plate takes the shape of a surface of the cover facing the moving plate.

Preferably, the moving means includes one or more rotation gears and a crank shaft connected to the rotating gear.

Preferably, the moving means includes a piston and a motor that vertically moves the piston.

Preferably, the sleeping inducer further includes a pad formed between the cover and the moving plate and capable of shrinking and expanding.

Preferably, the pad includes air therein.

Preferably, the moving means is a tube that is capable of shrinking and expanding.

Preferably, the moving means further includes air supply/removal means configured to supply air to the tube and to remove the air from the tube.

Preferably, the sleeping inducer further includes a controller configured to control a gap of moving time in the moving means.

Preferably, the cover is a doll or a pillow, or takes the shape of a pillow.

Technical subjects to be solved by the present disclosure are not restricted to the above-mentioned description, and any other technical problems not mentioned so far will be clearly appreciated from the following description by the skilled in the art.

Therefore, additional advantages, objects, and features of the disclosure will be set forth in part in the description which follows and in part will become apparent to those having ordinary skill in the art upon examination of the following or may be learned from practice of the disclosure. The objectives and other advantages of the disclosure may be realized and attained by the structure particularly pointed out in the written description and claims hereof as well as the appended drawings.

It is to be understood that both the foregoing general description and the following detailed description of the present disclosure are exemplary and explanatory and are intended to provide further explanation of the disclosure as claimed.

The sleeping inducer according to the present disclosure has an advantageous effect in that a means vertically movable in a doll or a pillow that is hugged in sleep is provided to induce a sleep state just like comfort and reality effect as those in a mother's bosom.

That is, the sleeping inducer according to the present disclosure can move like a lung that shrinks and expands in response to respiration to provide a sleep state excellent in comfort and reality effect like those of a mother’ bosom, whereby a user, an infant or a child easily distracted by noises or erratic sounds can be effectively and sufficiently induced to sleep without disapproval.

### BRIEF DESCRIPTION OF THE DRAWINGS

The accompanying drawings, which are included to provide a further understanding of the disclosure and are incorporated in and constitute a part of this application, illustrate embodiment of the disclosure and together with the description serve to explain the principle of the disclosure. In the drawings:

FIG. 1 is a plan illustrating a product applied with a sleeping inducer according to an exemplary embodiment of the present disclosure;

FIG. 2 is a lateral surface view of FIG. 1;

FIG. 3 is a mimetic diagram illustrating a basic configuration of a sleeping inducer according to the present disclosure;

FIG. 4 is a cross-sectional view illustrating a detailed configuration of a sleeping inducer according to a first exemplary embodiment of the present disclosure;

FIG. 5 is a cross-sectional view illustrating a sleep inducer formed with a piston as moving means according to an exemplary embodiment of the present disclosure; and

FIG. 6 is a cross-sectional view illustrating a detailed configuration of a sleeping inducer according to a second exemplary embodiment of the present disclosure.

### DETAILED DESCRIPTION OF THE DISCLOSURE

Hereinafter, exemplary embodiments of the present disclosure will be described in detail with reference to the accompanying drawings. In the drawings, sizes or shapes of constituent elements may be exaggerated for clarity and convenience.

In describing the present disclosure, detailed descriptions of constructions or processes known in the art may be omitted to avoid obscuring appreciation of the invention by a person of ordinary skill in the art with unnecessary detail regarding such known constructions and functions.

Accordingly, particular terms may be defined to describe the disclosure in the best mode as known by the inventors. Accordingly, the meaning of specific terms or words used in the specification and the claims should not be limited to the literal or commonly employed sense, but should be construed in accordance with the spirit and scope of the disclosure. The definitions of these terms therefore may be determined based on the contents throughout the specification.

Now, construction and operation of the linear oscillator according to the present disclosure will be described in detail with reference to the accompanying drawings.

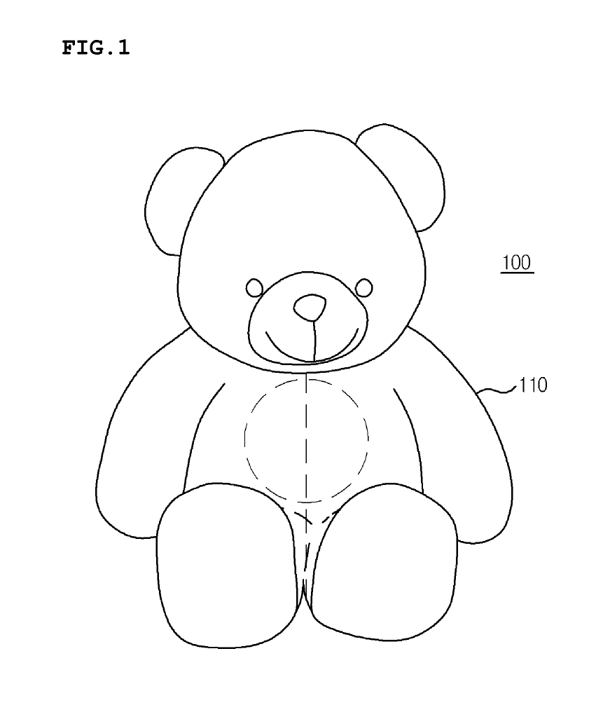

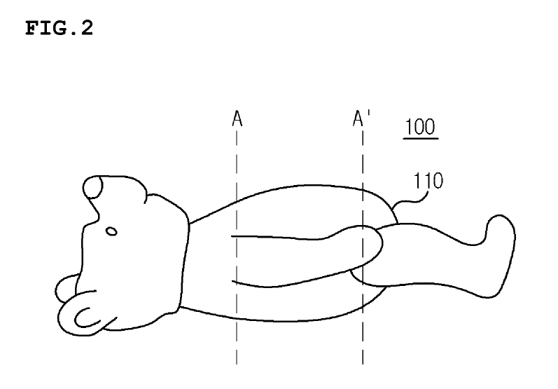

FIG. 1 is a plan illustrating a product applied with a sleeping inducer according to an exemplary embodiment of the present disclosure, and FIG. 2 is a lateral surface view of FIG. 1.

As illustrated in the drawings, the present disclosure relates to a device for inducing sleep. The device for inducing sleep (hereinafter referred to as ‘sleep inducer’) according to the present disclosure may take the shape of a product that can be hugged during sleep, or a product on which a head of a man can be placed during sleep.

That is, the sleeping inducer according to the present disclosure may be a product capable of moving along like a lung that is shrunk and expanded in response to respiration when a person respires during sleep.

To this end, the product preferably takes the shape of a doll or a pillow, whereby a sleep state just like being hugged and slept by a mom can be induced.

Thus, the sleeping inducer according to the present disclosure may be a pillow or a doll per se, or a device that can be included in the pillow or the doll, for example, where, in this particular case, the sleeping inducer according to the present disclosure is preferably contained in an area that can be most closely abutted to a user.

For example, as illustrated in FIGS. 1 and 2, the sleeping inducer according to the present disclosure is preferably mounted at an area taking a largest portion of a doll, i.e., a chest portion of the doll.

Now, although the exemplary embodiment of the present disclosure has illustrated and described a bear doll, the present disclosure is not limited thereto. For example, the sleeping inducer according to the present disclosure may be applied to a doll having a different shape and a pillow, or to a device particularly designed for sleep inducement.

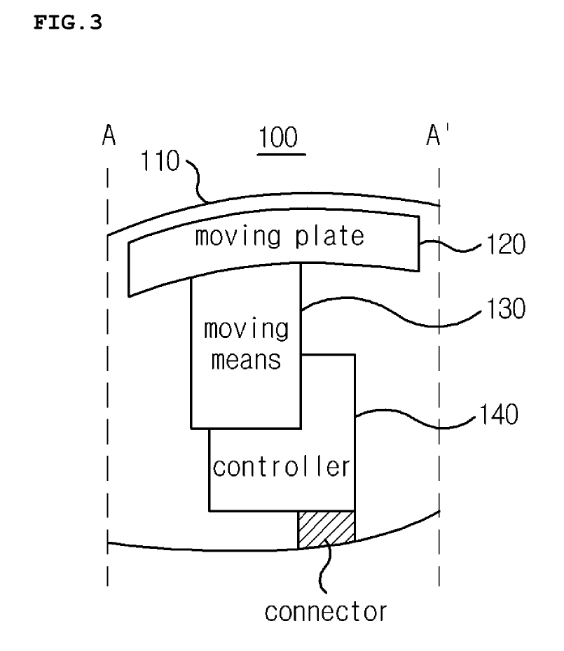

FIG. 3 is a mimetic diagram illustrating a basic configuration of a sleeping inducer according to the present disclosure, where the sleep inducer includes a cover (110), a moving plate (120) and a moving means (130).

The cover (110) is an external appearance encompassing the present disclosure, and may be a body or a housing. Therefore, the shape of the cover (110) is not particularly limited. For example, the cover (110) may be a doll or a pillow, or an appearance taking the shape of a doll or a pillow.

Preferably, the cover (11) is made of flexible material to easily transmit motion of the moving plate (120, described later) to the outside. For example, the cover (110) may be cloth of polymer having an excellent flexibility, or a metal plate clothed with a flexible material.

The moving plate (120) is disposed inside the housing encompassed by the cover (110), such that the moving plate (120) may be a plate or a sheet mounted right underneath the cover (110).

The moving plate (120) according to the present disclosure is vertically moved by the moving means (130, described later).

That is, the moving plate (120) according to the present disclosure vertically moves by being disposed right below the cover (110), and functions to lift up or lower down a cover (110) region corresponding thereto, whereby a partial portion of the cover (110) can have a movement to ascend and descend to the outside.

The moving plate (120) suffices to deform an exterior appearance of the cover (110) positioned thereon, such that there is no particular limit to the shape or material. However, the moving plate (120) preferably takes the shape corresponding thereto to be sufficient enough to lift up or lower down a large area of the cover (110). More preferably, the moving plate (120) has a size of that of a lung in response to the movement of a lung.

The moving means (130) functions to repeatedly and vertically move the moving plate (120). The vertical movement of the moving plate (120) can raise a part or an entire area of the cover (110) positioned thereon, whereby the sleep inducer according to the present disclosure can move just like a lung that is shrunk and expanded in response to respiration.

The method and structure of the moving means (130) comprised of a plate and a sheet that moves the moving plate (120) are not particularly limited. The moving means (130) is preferably included inside and fixed at the housing encompassed by the cover (110), and shape or material is not particularly limited, details of which will be described later.

The sleeping inducer according to the present disclosure may further include a control unit (140) for supplying power to the moving means (130) and controlling the moving means (130), and a connector for connecting to the outside.

As apparent from the foregoing, the sleeping inducer according to the present disclosure has an advantageous effect in that the sleeping inducer includes a vertically movable means inside a doll held during sleep or a pillow to sleep on to induce comfort and reality effect like those of a mother's bosom.

That is, the sleeping inducer according to the present disclosure can move like a lung that is shrunk and expanded responsive to respiration to provide an excellent sleep environment of comfort and reality effect like those of a mother's bosom, whereby a user, an infant or a child sensitive to or easily distracted by noises or erratic sounds can be effectively and sufficiently induced to sleep without any disapproval.

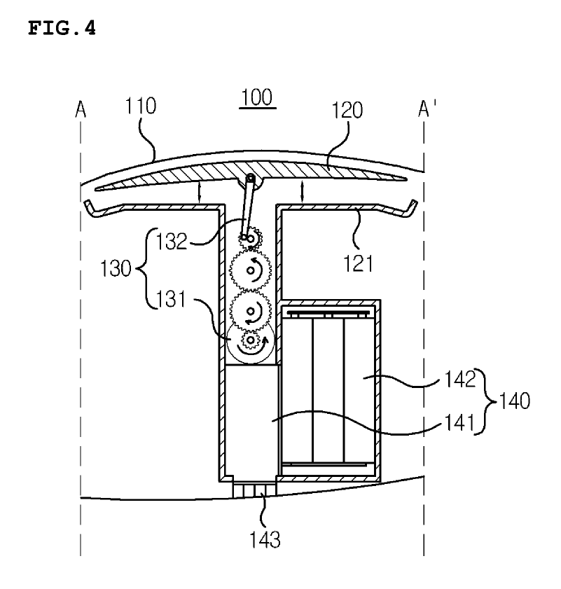

FIG. 4 is a cross-sectional view illustrating a detailed configuration of a sleeping inducer according to a first exemplary embodiment of the present disclosure.

As described in the above, the moving plate (120) according to the first exemplary embodiment of the present disclosure can move up and lower down a cover (110) area corresponding thereto.

To this end, the moving plate (120) may be distanced from the cover (110) or may be abutted to the cover (110).

That is, the moving plate (120) vertically moves to ascend or descend the cover (110) while being distanced apart from the cover (110) at a predetermined space, where the distance or gap between the moving plate (120) and the cover (110) is preferably within the range of 0.1 cm to 5.0 cm.

If the distance is deviated from the given range, movement of the cover (110) can be ineffectively small or great over a moving distance of the moving plate (120).

Furthermore, the moving plate (120) may be closely adhered to the cover (110), whereby the cover (110) can be directly moved in response to the motion of the moving plate (120). If the moving plate (120) is closely adhered to the cover (110), it is more preferable that the movement size of the cover (110) can be adjusted more accurately responsive to the moving distance of the moving plate (120).

Another characteristic of the present disclosure is that the moving plate (120) takes the shape corresponding to that of one surface of the cover (110) facing the moving plate (120). That is, if the cover (110) takes the shape of a plane, the moving plate (120) corresponding thereto may also take the shape of a plane, and if the cover (110) takes the shape of a curvature, the moving plate (120) corresponding thereto may also take the shape of a curvature.

As noted above, if the corresponding surface takes the same shape or a similar shape, the sleeping inducer according to the present disclosure can evenly move on a broader area of the cover (110) by the movement of the moving plate (120), whereby the sleeping inducer according to the present disclosure can have a movement similar to that of the lung.

Furthermore, the sleeping inducer according to the present disclosure is contained in the cover (110), and may further include a support unit (121) mounted underneath the moving plate (120).

The support unit (121) is disposed underneath the moving plate (120) and functions to support the moving plate (120) and to prevent the moving plate (120) from moving further down. The moving plate (120) is disposed thereon with the cover (110), such that upward support can be possible by the moving means (130) connected to the moving plate (120).

It is therefore preferable that there be the support unit (121) supporting the moving plate (120), if there is a void underneath the moving plate (120). Although FIG. 4 illustrates the support unit (121) as a plate corresponding to the moving plate (120), the configuration of the support unit (121) is not limited thereto. For example, the support unit (121) may be a material that fills up the void inside the cover (110).

At the same time, the moving means (130) according to the present disclosure may include at least one or more rotation gears (131) connected to a bottom surface of the moving plate (120), and a crank shaft (132).

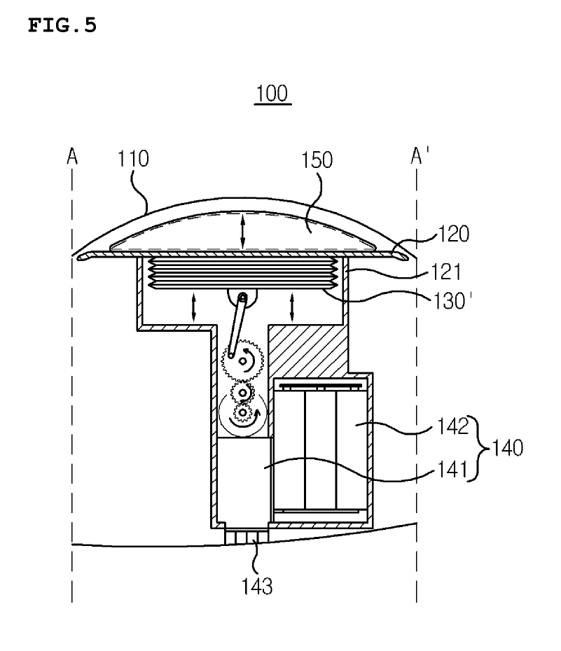

The crank shaft (132) functions to convert a reciprocating movement of the rotation gear (131) to a linear movement, and transmit the linear movement to the moving plate (120). Furthermore, as illustrated in FIG. 5, the moving means (130) may include one or more pistons (133) connected to the bottom surface of the moving plate (120), and a motor (134) vertically moving the piston (133).

If the vertically movable piston (133) is employed, it is possible to move an entire portion of the moving plate (120) more smoothly, and to further approach the movement of the lung, which is more adequate to the purpose of the present disclosure.

The sleeping inducer according to the present disclosure may further include a controller (141) as the control unit (140) for controlling the functions of the moving means (130), and a power supply unit (142) supplying power to the control unit (140) and the controller (141).

The sleeping inducer according to the present disclosure may still further include an external charging means and a connector (143) for connecting to a USB.

Preferably, the controller (141) control a time gap to allow the moving means (130) to vertically move the moving plate (120) approximately 10˜25 times, more preferably approximately 15˜20 times, for more realistic movement.

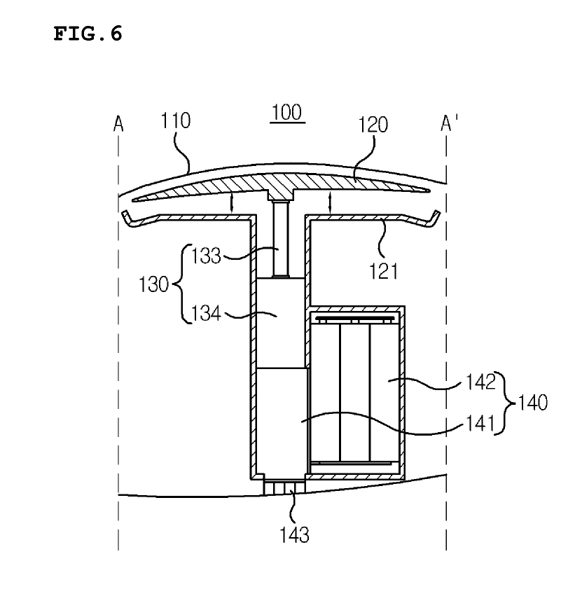

FIG. 6 is a cross-sectional view illustrating a detailed configuration of a sleeping inducer according to a second exemplary embodiment of the present disclosure.

Referring to FIG. 6, the sleeping inducer according to the second exemplary embodiment of the present disclosure may further include a pad (150). The pad (150) is contained in the cover (110), and is interposed between an upper surface of the cover (110) and a bottom surface of the moving plate (120) to shrink and to expand. Therefore, the moving state of the moving plate (120) can be more smoothly and effectively transmitted to a front surface of the cover (110).

To this end, the pad (150) more preferably contains air therein. That is, the pad (150) may be made of a rubber material like that of a balloon. If the flat moving plate (120) mounted thereunder moves, there is an effect that the air inside the pad (150) can be shrunk and expanded to allow evenly transmitting movement to an entire area of the cover (110) mounted thereon.

Referring to FIG. 6 again, the moving means (130) according to the present disclosure is contained in the cover (110), and may be a foldable tube (130′) that is shrunk and expanded underneath the moving plate (120). That is, air is poured into the foldable tube (130′) to move the moving plate (120) upward, and the air is discharged from the foldable tube (130′) to move the moving plate (120) downward.

To this end, the moving means (130) may further include an air supply/removal means (not shown) for supplying air to the tube (130′) and for removing the air from the tube (130′). As noted above, if the tube is used as moving means (130), it is possible to smoothly and slowly move the moving plate (120) and the cover (110), whereby an effect similar to the movement of a lung can be accomplished.

The above-mentioned sleeping inducer according to the present disclosure may, however, be embodied in many different forms and should not be construed as limited to the embodiment set forth herein. Thus, it is intended that embodiment of the present disclosure may cover the modifications and variations of this disclosure provided they come within the scope of the appended claims and their equivalents. While particular features or aspects may have been disclosed with respect to several embodiments, such features or aspects may be selectively combined with one or more other features and/or aspects of other embodiments as may be desired.

As apparent from the foregoing, the sleeping inducer according to the present disclosure has an industrial adaptability in that the sleeping inducer is capable moving like a lung that is shrunk and expanded in response to respiration when a person respires during sleep, to thereby induce a sleep state just like being hugged and slept by a mom.

What is claimed is:

1. A sleeping inducer, the sleeping inducer comprising: a cover; a moving plate included inside the cover and disposed underneath the cover; and moving means repeatedly and vertically moving the moving plate.

2. The sleeping inducer of claim 1, wherein the moving plate is distanced from the cover in the range of a gap of 0.1 cm to 5.0 cm.

3. The sleeping inducer of claim 1, wherein the moving plate is closely adhered to the cover.

4. The sleeping inducer of claim 1, wherein the moving plate takes the shape of a surface of the cover facing the moving plate.

5. The sleeping inducer of claim 1, wherein the moving means includes one or more rotation gears and a crank shaft connected to the rotating gear.

6. The sleeping inducer of claim 1, wherein the moving means includes a piston and a motor that vertically moves the piston.

7. The sleeping inducer of claim 1, further comprising: a pad formed between the cover and the moving plate and capable of shrinking and expanding.

8. The sleeping inducer of claim 7, wherein the pad includes air therein.

9. The sleeping inducer of claim 1, wherein the moving means is a tube that is capable of shrinking and expanding.

10. The sleeping inducer of claim 9, wherein the moving means further includes air supply/removal means configured to supply air to the tube and to remove the air from the tube.

11. The sleeping inducer of claim 1, further comprising a controller configured to control a gap of moving time in the moving means.

12. The sleeping inducer of claim 1, wherein the cover is a doll or a pillow, or takes the shape of a pillow.
